(19) United States
(12) Patent Application Publication
(10) Pub. No.: US 2011/0244448 A1
(43) Pub. Date: Oct. 6, 2011

(54) DNA DETECTING APPARATUS, DNA DETECTING DEVICE AND DNA DETECTING METHOD

(76) Inventors: Masataka Shirai, Higashimurayama (JP); Tomoharu Kajiyama, Higashiyamato (JP); Hideki Kambara, Hachioji (JP)

(21) Appl. No.: 13/058,934

(22) PCT Filed: Aug. 31, 2009
(86) PCT No.: PCT/JP2009/065205

§ 371 Date: Apr. 28, 2011

(30) Foreign Application Priority Data
Sep. 8, 2008 (JP) 2008-229980

(51) Int. Cl. C12Q 1/68; C12M 1/40

(52) U.S. Cl. 435/6.1; 435/287.2

(57) ABSTRACT

A chemiluminescence detecting apparatus includes a thing called a plate where many reaction chambers are one-dimensionally or two-dimensionally arranged, and enzymes packed in a gel and DNA-immobilized beads are simultaneously charged into the reaction chambers, thereby trapping a large amount of enzymes in the reaction chambers, improving enzyme activity and achieving high sensitive pyrosequencing.

### TECHNICAL FIELD

The present invention relates to an apparatus, device and method for DNA detection and further DNA base sequencing, and also to an apparatus, device and method characterized by using chemiluminescence for the detection.

### BACKGROUND ART

For the DNA base sequencing, a method based on the Sanger method and using gel electrophoresis and fluorescence detection has been widely used. In this method, many copies of a DNA fragment to be subjected to sequence analysis are first produced. By using these as a template, with the 5′ end of DNA being taken as a starting point, fluorescent label fragments of various lengths are produced by complementary strand synthesis. At this time, a small amount of fluorescent label terminator (nucleic acid analog) is added to four types of nucleic acid, which are substrates for complementary strand synthesis, thereby producing a group of DNA fragments having various lengths and having fluorescent labels of different wavelengths according to the base type of the 3′ end. These are subjected to gel electrophoresis so that the difference in length is identified by the difference of the 1 base and luminescence from each fragment is detected. From luminescent wavelength color, the type of DNA end base of each DNA fragment under the measurement is found. Since DNAs sequentially pass through a fluorescence detecting unit in ascending order of the fragment length, the type of end base can be found from the shortest DNA by measuring fluorescent color. In this manner, the sequencing is carried out. The fluorescent DNA sequencer like this has been widely available and also achieved a great success in human genome analysis. Regarding this method, a technology has been disclosed in which the number of analyses per machine is increased by using a plurality of glass capillaries having an inner diameter of about 50 μm and further utilizing a method such as end detection (for example, refer to Non-Patent Document 1).

Also, in recent years, a sequencing method through stepwise chemical reaction typified by pyrosequencing (for example, refer to Patent Documents 1 and 2) has been attracting attention in view of convenience of handling, and the following is a brief description thereof. Primers are hybridized with a target DNA strand, and four nucleic acid substrates for complementary strand synthesis (dATP, dCTP, dGTP and dTTP) are sequentially added one by one into a reaction solution for a complementary strand synthesis reaction. When a complementary strand synthesis reaction occurs, a DNA complementary strand extends to produce pyrophosphoric acid (PPi) as a byproduct. PPi is converted to ATP by the catalytic action of a coexisting enzyme, and reacts under the coexistence of luciferin and luciferase to produce luminescence. By detecting this light, the incorporation of the added substrates for complementary strand synthesis into the DNA strand is found, so that the sequence information of complementary strands, that is, the sequence information of the target DNA strand can be found.

In this method, by using a flow cell including many reaction chambers, simultaneous sequence analyses of many DNA fragments can be performed, that is, throughput can be increased, and an example of significantly increasing the number of analyses by applying this method has been reported (for example, refer to Non-Patent Document 2). In this example of application, a flow cell having a reaction plate where a plurality of minute reaction chambers are arranged on a plane is used. In this reported example, many sepharose-made beads having a diameter of approximately 35 μm where target DNA strands are immobilized for each sequence are prepared for analysis. Approximately 10⁸ DNAs of the same type are immobilized on each sepharose bead. After primers are hybridized with these DNAs, one bead is injected so that up to one sequence of DNA is immobilized on each reaction chamber (if the number of sequences of DNA in the chamber is one, the number of beads in the chamber can be plural). Also, microbeads each having a diameter of 2.8 μm on which enzymes for chemiluminescence detection (luciferase and ATP sulfrylase) are immobilized are charged into the reaction chambers. These beads are charged by introducing a solution containing the beads to the flow cell and spinning down the beads by a centrifuge.

Here, the apparatus described in Non-Patent Document 2 is more specifically described with reference to FIG. 2. FIG. 2 depicts a sectional view of a flow cell including many reaction chambers according to a conventional example (Non-Patent Document 2). In FIG. 2, 101 denotes a plate for chemiluminescence reaction, on which a plurality of reaction chambers 102 are formed. Inside of each of these reaction chambers 102, one bead 103 on which a DNA to be subjected to the sequence analysis is immobilized is placed. In Non-Patent Document 2, a sepharose bead having a diameter of about 34 μm is used as this bead 103, and the diameter of the reaction chamber 102 is set at 44 μm, thereby allowing only one bead to be placed in one reaction chamber. Furthermore, microbeads 104 each having a diameter of 2.8 μm on which enzymes for chemiluminescence (in Non-Patent Document 2, luciferase and ATP sulfrylase) are immobilized are charged together with the DNA-immobilized bead 103 into the reaction chamber 102 by the centrifugal apparatus. Next, a top plate 105 having a transparent window area for measuring the chemiluminescence is fixed so as to face the plate 101 with a certain gap (about 0.3 mm). This gap serves as a flow path for reagents. By sequentially supplying nucleic acid substrates required for four-type complementary strand reaction and substrates required for chemiluminescence (luciferin and APS (Adenosine 5′-phosphosulfate)) through the path, complementary strand reaction and its accompanying chemiluminescence reaction occur, and luminescence from the inside of the reaction chambers 102 are measured by an image pickup device such as a CCD.

In the DNA base sequence analysis, four types of nucleic acid substrate for complementary strand synthesis (dATP, dCTP, dGTP and dTTP) for extension reaction are sequentially introduced from an upstream of the flow cell to perform the complementary strand synthesis reaction. When the complementary strand synthesis reaction proceeds, PPi is produced and then converted to ATP for the luciferase reaction, and the chemiluminescence occurring at this time is measured. Several reports have been issued about such apparatuses that use many reaction chambers to detect chemiluminescence and fluorescence. Examples include the case where, in place of immobilizing DNA on a bead, a DNA probe is immobilized on one end face of an optical fiber plate and is bonded to circular nucleic acid templates, thereby performing the sequencing and polymorphism analysis with chemiluminescence (for example, refer to Patent Document 2) and the case where the optical fiber plate is etched to remove the center portion of the fiber to produce reaction chambers to configure a picotiter plate (hereinafter abbreviated as a “plate”) for use as a part of a flow cell (for example, refer to Patent Document 3). Furthermore, for example, Patent Document 4 discloses a plate added with a membrane or the like for reducing contamination due to diffusion of a substance to be generated, specifically, PPi, in a horizontal direction inside each reaction chamber in this plate.

Also, Non-Patent Documents 4 and 5 disclose methods of immobilizing an enzyme for chemiluminescence detection in a gel. Further, Non-Patent Document 6 discloses photoreactive polyvinyl alcohol as a photoreactive polymer material that gels with photoirradiation.

Furthermore, Non-Patent Document 7 discloses an example in which PPDK (Pyruvate Orthophosphatase Dikinase) is used for pyrosequencing as an enzyme for chemiluminescence detection in place of ATP sulfrylase.

### PRIOR ART DOCUMENTS

### Patent Documents

Patent Document 1: International Publication Pamphlet No. 98/28440
Patent Document 2: International Publication Pamphlet No. 01/020039
Patent Document 3: International Publication Pamphlet No. 03/004690
Patent Document 4: Japanese Patent Application Publication No. 2003-515107

### Non-Patent Documents

Non-Patent Document 1: Anal. Chem., Vol. 72, 2000 reaction chamber pp. 3423-3430
Non-Patent Document 2: Margulies M, et al., “Genome sequencing in microfabricated high-density picolitre reactors.”, Nature, Vol. 437, Sep. 15; 2005, pp. 376-380 and Supplementary Information s1-s3
Non-Patent Document 3: Electrophoresis, Vol. 24, 2003, pp. 3769-3777
Non-Patent Document 4: Kunio Ohyama, Kaoru Omura, Yoshihiro Ito, Allergology International, Vol. 54, 2005, pp. 627-631
Non-Patent Document 5: Yoshihiro Ito, Hirokazu Hasuda, Makoto Sakuragi, Saki Tsuzuki, Acta Biomaterialia, Vol. 3 (6), 2007, pp. 1024-1032
Non-Patent Document 6: Yoshihiro Ito, Masayuki Nogawa, Mineko Takeda, Toru Shibuya, Biomaterials, Vol. 26, 2005, pp. 211-216
Non-Patent Document 7: G. Zhou, T. Kajiyama, M. Gotou, A. Kishimoto, S. Suzuki and H. Kambara, Anal. Chem. Vol. 78 (13), 4482 (2006)

### SUMMARY OF THE INVENTION

### Problems to be Solved by the Invention

In a DNA detecting apparatus including many reaction chambers and using chemiluminescence detection, luminescence signals output from the reaction chambers on the plate are gathered to an image pickup device for observation. In this case, throughput (the number of genes that can be measured and sequenced at one time) can be improved at low cost by increasing the number of beads measurable by one apparatus. To achieve this, appropriately setting a magnification of an optical system and setting a one-to-one correspondence between beads and pixels can improve the throughput.

However, since light-gathering efficiency from the reaction chambers to the image pickup device for chemiluminescence is decreased in inverse proportion to the square of the magnification, increasing the magnification invites a significant decrease in sensitivity. To measure light most efficiently, it will be best to set a lens system at unity magnification (=1×) (from Lagrange's Law, it is known that luminescence density cannot be improved by using a geometrical optical system).

Also, since read noise is proportional to the number of pixels, read noise can be reduced by decreasing the number of pixels forming an image of one bead as much as possible with the magnification of the optical system being set at 1:1.

In practice, however, a practical pixel size is so small as several microns (practically, three to ten microns). Since the diameter of the reaction chamber disclosed in Non-Patent Document 2 is so large as 44 μm, when it is assumed that the optical system has a unity magnification, in order to match the pitches of the reaction chambers and the pixels to make a one-to-one correspondence between the reaction chambers and the pixels for measurement, the pixels have to be made large or the reaction chambers have to be made small.

If the number of beads is increased by setting the number of pixels constant and making the pixels large to achieve the one-to-one correspondence described above, throughput can be improved. However, the chip area of the image pickup device becomes large, thereby increasing manufacturing cost and also increasing dark current noise (thermal noise) to decrease performance. Therefore, this is not practical.

To get around this, a decrease in size of the reaction chamber and the beads for immobilizing DNAs is desired. However, as for a practical pixel size, the reaction chambers suitable for several microns each have a diameter on about 6 μm, and the beads for use also each have a diameter of about 5 microns at best. Since such beads cannot retain a sufficient amount of DNAs on their surfaces, they have the problem of insufficient detection sensitivity. Moreover, even if larger reaction chambers are used, an improvement in signal intensity is required to obtain an accurate signal.

For the improvement of detection sensitivity, it is effective to improve enzyme activity per unit volume in a reaction chamber (volume excluding a bead). The reason is as follows. That is, when the diameter of the DNA-immobilized bead in the reaction chamber is set to 1/p (p>1), the number of DNA molecules on the DNA-immobilized bead becomes smaller in proportion to 1/p², while the volume of the enzyme-immobilized bead to be charged becomes smaller in proportion to 1/p³. In other words, enzyme activity relatively decreases. This poses no problem if enzyme activity is sufficient with the size of a conventional DNA-immobilized bead, but in practice, even when the amount of DNA immobilization is small (about 10⁶ molecules/bead) in the conventional art, the situation is such that activity decreases in proportion to the enzyme amount. That is, it can be understood that, for making the DNA-immobilized beads smaller, it is required to significantly improve the enzyme activity per unit volume.

The present invention has been devised in view of the situation as described above, and it provides a DNA detecting device capable of improving enzyme activity per unit volume in each reaction chamber and sufficiently ensuring detection sensitivity of chemiluminescence, a DNA detecting apparatus including the device, and a DNA detecting method.

### Means for Solving the Problems

First, factors having an influence on detection sensitivity are summarized and examined. These factors include: 1) light-receiving efficiency of a detecting system; 2) performance of a detection element; 3) the number of DNAs immobilized on target beads; 4) improvement in efficiency of a luminescent enzyme reaction using PPi producing from a complementary strand synthesis reaction; and others.

That is, if these factors are improved, detection sensitivity to chemiluminescence can be improved. Among these factors, 1) and 2) can be addressed by setting the image-forming optical system at 1:1 and using a high-sensitivity cooled CCD or a CCD equipped with an electron multiplying function. Also, as for 3) the number of DNAs immobilized on the surface of the beads, it is approximately determined by the surface area, and therefore by the diameter of the bead. For example, by covering the surface with a polymer brush or making the surface rough to increase an effective area, the amount of DNAs that can be immobilized can be increased by several percents.

Therefore, for the further improvement in the detection efficiency, 4) improvement in efficiency of a luminescent enzyme reaction using PPi produced from a complementary strand synthesis reaction has become important. Note that the enzyme reaction for use herein is a cycle reaction as described in detail in the embodiments. That is, PPi produced in a complementary strand synthesis reaction is converted to ATP, and light is emitted by a luciferase reaction. As a byproduct, PPi is again produced and is again converted to ATP to contribute to a luminescence reaction, but it is degraded by a coexisting degrading enzyme if efficiency (activity) in an enzyme reaction per unit volume is low. Although this luminescence cycle is desirably repeated again and again by increasing the enzyme concentration, if an enzyme solution is added to the reaction chamber, the enzyme itself is lost in a cleaning process accompanied by replacement of a reactive substrate.

On the other hand, if an enzyme immobilized on a bead is charged as in the conventional example, the enzyme is retained only on the bead surface. Therefore, there is a problem that the density of the enzyme cannot be increased much from a three-dimensional point of view. To get around this, in the present invention, an enzyme in a solution form is efficiently injected into the reaction chamber and a gel matrix is used so as to prevent the drainage thereof in a cleaning process, thereby overcoming the problems in the conventional example. In other words, by the improvement regarding 4) above, the sensitivity can be increased by one order of magnitude or more.

More specifically, in the structure of a flow cell portion of the apparatus, the reaction chambers 102 are formed on the plate 101, and the DNA-immobilized beads 103 are charged thereinto. Then, after a reagent containing an enzyme and a photoreactive polymer mixed together is dropped from above them, spin coating is performed to form a polymer charged portion as indicated by 106 in FIG. 1. Thereafter, drying is performed for a certain period of time to the extent that enzyme activity in the reaction chamber is not much decreased, and then, the photoreactive polymer charged portion is gelled by the irradiation of ultraviolet rays. With the gelling in the reaction chamber, drainage of the enzyme was able to be suppressed as long as only a portion near the surface was sufficiently cured. By this means, several-fold enzyme activity was able to be obtained compared with the case of immobilization on the microbead surface (refer to a first embodiment). The reason for this achievement is that, since an area where a gel is to be charged is limited to the inside of the reaction chamber, deformation and drainage of the gel can be prevented even if the strength of the gel is not sufficient. Since the gel is charged into only the reaction chamber, the improvement in enzyme activity can be achieved by removing the photoreactive polymer in an area other than the reaction chamber with the use of a spin coater before photo-curing.

Next, how much enzyme activity can be improved per unit volume by immobilizing the enzyme on the gel instead of immobilizing the enzyme on the microbead surface is described. When the enzyme is immobilized on the microbead, only one molecule can be immobilized on an 8 nm square area at most. Thus, on a microbead having a diameter of 2.8 μm, enzymes of 4×10⁵ molecules can be immobilized.

On the other hand, the volume inside the reaction chamber where beads can be charged is 15 pL. When it is assumed that beads can be charged in a closest-packed manner (74%), approximately 1000 beads can be packed in the reaction chamber. Therefore, 4×10⁸ enzyme molecules are immobilized inside the reaction chamber. On the other hand, in the structure of the present invention, since a photoreactive polymer solution with luciferase having a molecular weight of 60000 and having an enzyme concentration of 20 mg/mL can be prepared, the number of enzyme molecules in the reaction chamber can be 3×10⁹.

Therefore, it can be understood that the number of enzymes in the reaction chamber can be improved approximately by one order of magnitude. When the enzyme is immobilized on the microbead surface, activity cannot be kept depending on the immobilizing direction in some cases. Thus, it can be thought that more difference in activity occurs in practice. Needles to say, the same goes for other enzyme immobilization.

The above is summarized as follows. That is, for solving the problems described above, a DNA detecting apparatus according to the present invention is a DNA detecting apparatus which detects chemiluminescence from a plurality of reaction chambers for DNA detection or DNA analysis, and the apparatus includes: a flow cell having a plate on a surface of which a plurality of reaction chambers are one-dimensionally or two-dimensionally arranged; and light detecting means having a plurality of pixels and detecting chemiluminescence. Furthermore, one or plural beads having up to one type of DNA immobilized thereto and a gel containing at least an enzyme required for chemiluminescence detection are charged into each of the reaction chambers. Here, the gel charged into the reaction chamber is gelled and solidified by light irradiation at an opening of the reaction chamber. Also, the enzyme contained in the gel includes luciferase. Furthermore, the gel charged into the reaction chamber contains an azido group in a polymer gelled by light irradiation. Note that each of the reaction chambers has a convex structure therein, only one DNA-immobilized bead can be charged into the reaction chamber, and an area where the gel can be charged is present inside the reaction chamber.

A DNA detecting device according to the present invention is a DNA detecting device used for detecting chemiluminescence from a plurality of reaction chambers, and the device includes: a flow cell having a plate on a surface of which a plurality of reaction chambers are one-dimensionally or two-dimensionally arranged. Also, a gel containing an enzyme required for the detection of chemiluminescence is charged in the reaction chambers, and in a state where the gel is charged, a space (concave portion) for charging a DNA-immobilized bead remains inside the reaction chamber at a portion closer to an opening of the reaction chamber than to a portion where the gel is charged. Note that the reaction chamber formed on the plate may have a tapered shape whose opening is wider than a bottom portion.

A DNA detecting method according to the present invention is a DNA detecting method which detects chemiluminescence from a plurality of reaction chambers for DNA detection or DNA analysis, and the method includes the steps of: charging DNA-immobilized beads into a plurality of reaction chambers which are one-dimensionally or two-dimensionally arranged on a plate; charging a photoreactive polymer containing an enzyme required for chemiluminescence detection into the reaction chambers; gelling the photoreactive polymer by light irradiation; and introducing a reagent relating to a luminescence reaction onto the reaction chambers to cause chemiluminescence and detecting this chemiluminescence. The DNA detecting method further includes the step of: removing the photoreactive polymer remaining in a portion other than the reaction cambers on the plate, and after the superfluous photoreactive polymer is removed, the step of gelling the photoreactive polymer by the light irradiation is performed.

Another DNA detecting method according to the present invention is a DNA detecting method which detects chemiluminescence from a plurality of reaction chambers for DNA detection or DNA analysis, and the method includes the steps of: charging a photoreactive polymer containing an enzyme into a plurality of reaction chambers which are one-dimensionally or two-dimensionally arranged on a plate; gelling the photoreactive polymer by light irradiation; in a state where the gel is charged, charging DNA-immobilized beads into a space (concave portion) inside the reaction chamber at a portion closer to an opening of the reaction chamber than to a portion where the gel is charged; and introducing a reagent on the reaction chambers to cause chemiluminescence and detecting this chemiluminescence.

In the above-described DNA detecting method, the step of charging the photoreactive polymer into the reaction chambers and the step of gelling the photoreactive polymer by light irradiation may be performed a plurality of times.

Further features of the present invention will become apparent below from the embodiments for carrying out the present invention and the attached drawings.

### Effects of the Invention

According to the present invention, the enzymes are retained with three-dimensionally high density in the reaction chamber, so that the enzyme reaction can be efficiently performed. Therefore, it is possible to repeatedly perform a reaction of converting PPi produced from complementary strand synthesis to ATP to perform a luminescence reaction and a luminescence reaction by converting PPi produced again as a byproduct of the previous luminescence reaction to ATP again.

Also, since the cycle of enzyme reaction can be efficiently repeated to significantly increase a total amount of luminescence, the detection sensitivity can be improved. As a result, even when a small amount of DNAs immobilized on small beads are used, it is possible to detect chemiluminescence from the beads and also achieve the DNA base sequencing.

Furthermore, since the reaction chamber and the detection element has a 1:1 relation in the apparatus, it also has an advantage that a large amount of DNA reagent can be analyzed in parallel by using a small and inexpensive apparatus.

### BRIEF DESCRIPTIONS OF THE DRAWINGS

FIG. 1 is a sectional view of a flow cell (detecting device) in a first embodiment of the present invention;

FIG. 2 is a sectional view of a flow cell in a conventional art;

FIG. 3 is a drawing depicting a schematic structure of a chemiluminescence detecting system in the present invention;

FIG. 4 is a structural drawing of the flow cell;

FIG. 5 is a drawing depicting a flow-cell fabricating method in the first embodiment;

FIG. 6 is a drawing depicting an example of luminescence image at the time of pyrosequencing;

FIG. 7 is a graph depicting an example of the results of performing pyrosequencing;

FIG. 8 is a graph depicting a ratio in chemiluminescent intensity of a conventional method and the present invention (first embodiment);

FIG. 9 is a drawing depicting a sectional view of a flow cell according to a second embodiment of the present invention;

FIG. 10 is a drawing depicting a method of creating gel-coated beads in a third embodiment;

FIG. 11 is a drawing depicting a sectional view of a flow cell according to the third embodiment; and

FIG. 12 is a drawing depicting a sectional view of a flow cell according to a fourth embodiment.

### BEST MODE FOR CARRYING OUT THE INVENTION

In the present invention, for improving enzyme activity per unit volume, enzymes are trapped in the reaction chamber by using a gel. At this time, in order to prevent drainage of the gel from the reaction chamber, the surface of the gel is cured.

Regarding the gel curing, Non-Patent Documents 4 and 5 disclose the methods of immobilizing protein such as an antibody by using a photoreactive polymer. In this method, unlike the case of immobilization on the bead surface, an area where beads cannot be immobilized such as the inside of the microbeads is not present, and the number of enzyme molecules that can be immobilized per unit volume of the inside can be increased.

In Non-Patent Documents 4 and 5, a film made of gel is formed on an inner wall of a flow path, and enzymes are trapped in that film. In this case, after a photoreactive polymer and the enzyme are mixed together, sufficient drying and irradiation of ultraviolet light with a sufficient intensity for a sufficiently long period of time are necessary. However, there is a problem that, as the drying and irradiation of ultraviolet light are performed for a longer time, activity of the trapped enzyme decreases more. On the other hand, if the drying and irradiation of ultraviolet light are not sufficient, curing is not sufficient, and the gel film comes off from the inner wall of the flow path. Also in the case of using a photoreactive polymer described in Non-Patent Document 6, the above problem is mitigated to some extent, but this mitigation is insufficient.

In the present invention, as will be described further below, a polymer is charged into a concave portion, that is, a reaction chamber and only the surface of the opening of the chamber is gelled. Therefore, irradiation of ultraviolet light until the entire polymer is gelled (cured) is not necessary. In the first place, in the methods of Non-Patent Documents 4 to 6, an idea of charging a gel into a reaction chamber is not present. This is based on the thought that a reaction with a reagent does not proceed if a gel substance is charged into the reaction chamber.

However, the inventors of the present invention found that, even when the surface of the photoreactive polymer is gelled, if the pore size of the gel is sufficiently larger than the molecule size of the reagent and is sufficiently smaller than that of enzymes such as luciferase and PPDK, there is no problem in reaction.

In the following, embodiments of the present invention will be described with reference to the attached drawings. However, it should be noted that these embodiments are merely examples for achieving the present invention and are not meant to be restrictive.

Also, in the drawings, common structures are provided with the same reference numerals.

### First Embodiment

(1) Overview of Pyrosequence

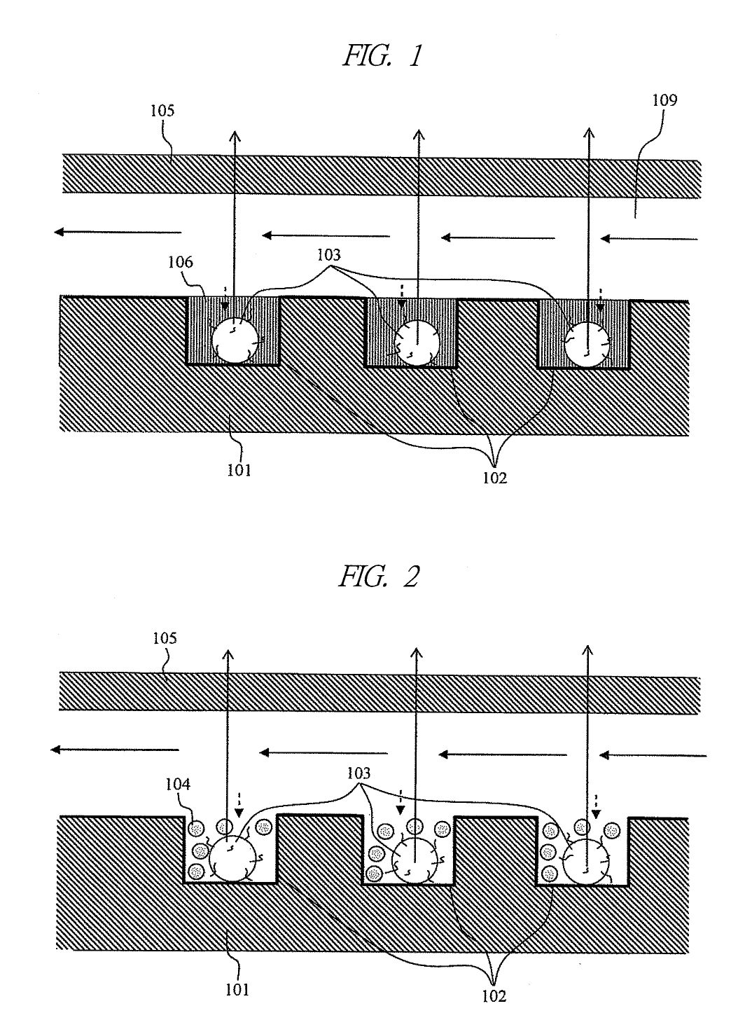

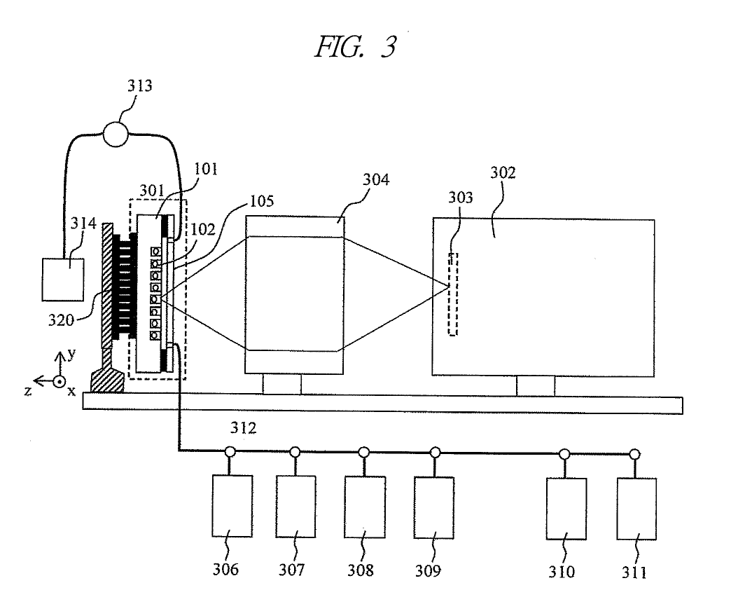

First, an overview of pyrosequencing in the present embodiment is described. FIG. 1 depicts a sectional view of a flow cell, and FIG. 3 depicts a structural diagram of the entire apparatus.

As depicted in FIG. 1, a target DNA is immobilized on a bead 103, and is retained in a reaction chamber 102. In the reaction chamber 102, enzymes retained in a gel matrix 106 are contained. An upper portion of the reaction chamber is opened, and reactive substrates are supplied in a solution state. The reactive substrate solution contains dNTP which is a substrate for a DNA complementary strand synthesis reaction, AMP which is a substrate for an ATP producing reaction, luciferin which is a substrate for a luminescence reaction and others.

A primer and a complementary strand synthesis enzyme are attached to a DNA reagent. There are four types of nucleic acid substrate dNTP, and these are sequentially added. After one substrate is added, a reaction flow path 109 and the reaction chambers 102 are cleaned with a cleaning fluid containing a degrading enzyme apyrase that degrades dNTP to remove the previous reactive substrate, and then the next reactive substrate is added.

When the added reactive substrate is complementary to a DNA strand, complementary strand synthesis occurs, and one complementary DNA strand extends with the primer as a base, and PPi is produced as a byproduct. With the action of PPDK, PPi is reacted with AMP to produce ATP. With the action of luciferase, ATP is reacted with luciferin to cause a luminescence reaction and also produce PPi and AMP as byproducts. PPi is again used for ATP producing reaction.

In the reaction chamber 102, a small quantity of the degrading enzyme apyrase coexists. When dNTP where the apyrase remains is degraded, ATP is also degraded. By this means, ATP caused by impurities can be degraded to suppress background luminescence, but ATP produced by the actual DNA complementary strand synthesis is also degraded. Thus, a luminescence cycle reaction is not infinitely repeated. It is required to perform many luminescence reaction cycles on average before the degrading reaction occurs. To this end, it is important to retain luminescence-related enzymes (PPDK and luciferase) with high density in the reaction chamber. To achieve this, a gel matrix is used in the present invention.

Also, in order to prevent drainage of the DNA-immobilized beads to the outside of the reaction chambers charged with the gel solution, the beads for use preferably have a specific gravity greater than that of the gel. Specifically, zirconia beads are used, but needless to say, other materials may be used.

(2) Structure of Chemiluminescence Detecting Apparatus

FIG. 3 is a drawing depicting a schematic structure of a chemiluminescence detecting apparatus in the present invention. In the chemiluminescence detecting apparatus, DNA-immobilized beads are charged into the reaction chambers 102 on the plate 101, and then a photoreactive polymer containing enzymes is charged so as to fill an area of each reaction chamber other than the beads. Subsequently, the photoreactive polymer is irradiated with ultraviolet light (including light having a wavelength of 300 nm) to cause the gelation to immobilize the enzymes. In this manner, the enzymes can be immobilized in the reaction chamber with higher density than that of the conventional example where enzymes are immobilized on microbeads and are charged into a reaction chamber (Non-Patent Document 2), and even when the number of DNA molecules on the beads is small, chemiluminescence associated with base extension can be measured and sequencing can be achieved.

In FIG. 3, the chemiluminescence detecting apparatus is a system of measuring chemiluminescence in the reaction chambers 102 on the plate 101. The chemiluminescence detecting apparatus includes a flow cell 301 made up by combining the plate 101 and a top plate 105 facing the plate together, an imaging camera 302 for obtaining, as image data, chemiluminescence occurring by causing the reagent to flow inside the flow cell to introduce the reagent molecules into the reaction chambers by the nucleic acid, and an optical system where a luminescence image from the reaction chamber 102 is formed on an image pickup device 303 such as a cooled CCD device inside the camera.

As the optical system, a tandem lens system (where the tips of two lenses are united and connected together and are fixed with their shooting distance being set at infinity for both of two) 304 capable of obtaining an erect image with a unity magnification can be used. By this means, a unity magnification is easily achieved, and luminescence can be most efficiently gathered to the image pickup device.

Also, the chemiluminescence detecting apparatus includes a system for delivering liquid reagents to the reaction chambers 102. More specifically, the apparatus includes reagent vessels 306 to 309 each accommodating the four types of nucleic acid substrate (for example, four types including dATP, dGTP, dCTP and dTTP) to sequentially dispense the reagent to the flow cell, a cleaning reagent vessel 310 accommodating a cleaning reagent for cleaning the flow cell after extension reaction measurement, a conditioning reagent vessel 311 accommodating a conditioning reagent for washing away residues of cleaning reagent components in the chambers after cleaning, an injecting unit (a selection valve 312 and a pump 313 for handling the reagents) for selectively injecting them to a flow cell side, a waste-fluid bottle 314 and others.

Furthermore, in order to set the temperature of the reagent solution inside the flow cell at an optimum temperature for pyrosequence, the chemiluminescence detecting apparatus is provided with a Peltier element 320, a thermistor and a temperature controller for controlling the Peltier element based on the temperature measured by the thermistor. Also, in order to reduce dark current noise of the image pickup (CCD) device 303, the image pickup device 303 is cooled to −57° C. This cooling temperature is determined so as to obtain a sufficient S/N ratio in accordance with the intensity of chemiluminescence. On the other hand, the temperature of the plate to be controlled by the Peltier element 320 is set at an optimum temperature for chemiluminescence, for example, at 37° C. in this case. Although this temperature also varies depending on the enzyme for use, it is set at an optimum temperature for KF and luciferase, which are polymerases.

(3) Structure of Flow Cell (Chemiluminescence Detecting Device)

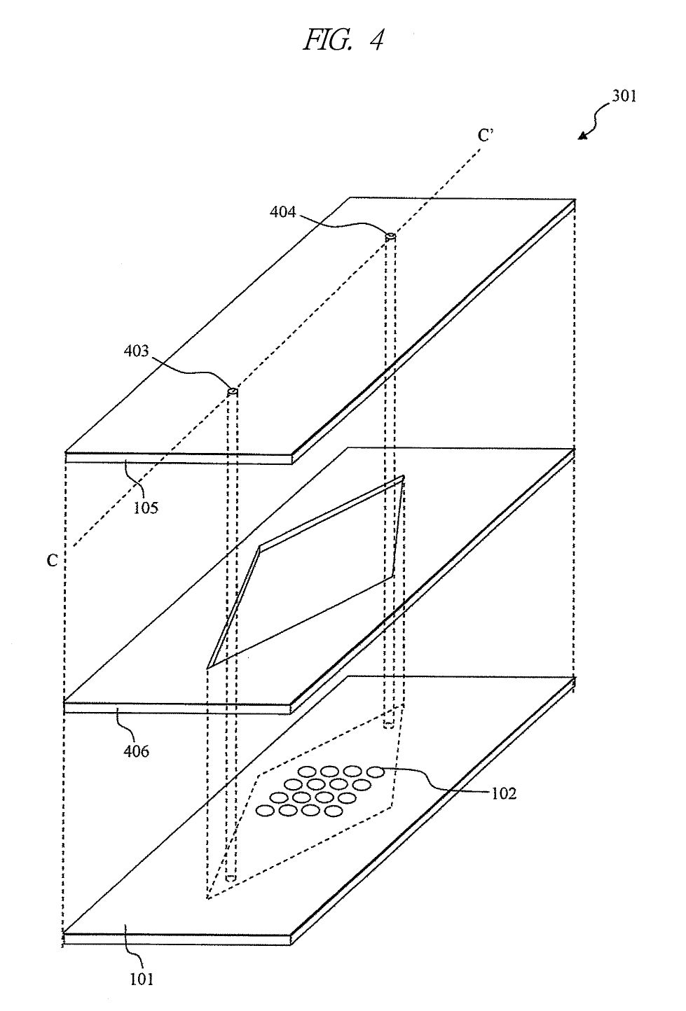

Next, the Structure of the Flow Cell 301, which is a device of detecting chemiluminescence, is described with reference to FIG. 4. The flow cell 301 includes the plate 101 having on its surface the plurality of reaction chambers (concave portions) 102 for retaining DNA-immobilized beads, a reagent inflow port 403, a reagent discharge port 404, the top plate 105 having a reagent injection port provided as required, and a spacer 406 forming a flow path.

FIG. 1 depicts a sectional view of the flow cell 301 along the line C-C′ in FIG. 4. As depicted in FIG. 1, a reagent flows inside the flow path 109 formed between the top plate 105 and the plate 101, and at this time, the required reagent is supplied to the reaction chambers 102. When the supplied base causes an extension reaction, chemiluminescence reaction occurs, and this is detected by the image pickup device. At this time, the beads 103 on which DNAs to be analyzed are immobilized are inserted in the reaction chambers 102, and also a gel 106 containing enzymes for chemiluminescence (luciferase and ATP sulfrylase or PPDK) is charged into the reaction chambers 102. Diffusion of reagent molecules such as the nucleic acid substrates occurs through cavities of a polymer forming the gel 106.

(4) Method of Fabricating Flow Cell Containing Enzyme-Added Gel (Specific Example)

A proper amount (in this example, 0.5 mg (approximately 16000 beads)) of DNA-immobilized beads is measured and taken, and the beads are injected into 50 μL of a bead incubation reagent described in Table 1 so that the DNAs on the beads are reliably bonded to polymerase (DNA polymerase l exo-klenow) and are reacted by a rotator for 3 minutes at ambient temperature.

TABLE 1
Bead incubation reagent (pH = 7.5)

| | | |
|---|---|---|
| Tricine | 50.0 | mM |
| EDTA | 1.0 | mM |
| MgAc | 10.0 | mM |
| DTT | 1.0 | mM |
| Klenow(exo-) | 0.2 | U/uL |
| SSB | 10.0 | ug/uL |
| Apyrase | 0.4 | U/mL |
| PPase | 0.1 | U/mL |

Apyrase and PPase in this reagent are mixed onto the bead surface and in polymerase reagent in order to degrade ATP and PPi that may cause background luminescence at the time of pyrosequence.

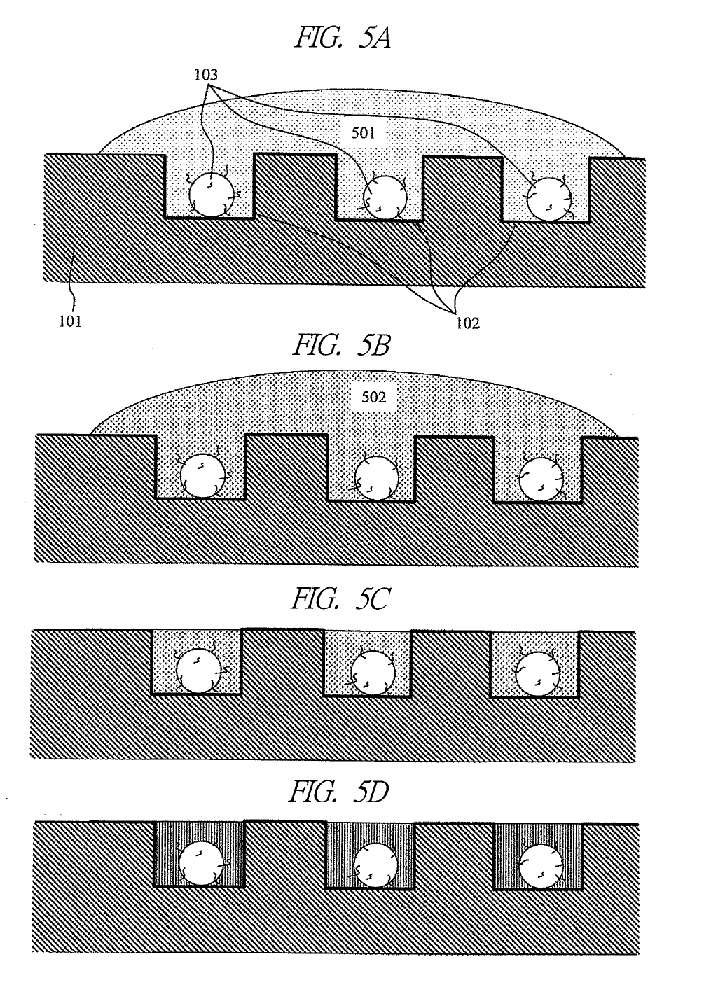

Next, on the plate 101, a 1×C buffer 501 described in Table 2 is dropped as depicted in FIG. 5A, thereby completely deaerating air holes inside the reaction chambers 102. Here, the DNA-immobilized beads described above are injected into the 1×C buffer 501 in FIG. 5A and immersed in the reaction chambers by using the fact that the specific gravity of the zirconia beads is as large as about 6. DNA-immobilized beads failing to be injected into the reaction chambers 102 are moved back and forward by tilting the flow cell so as to be completely inserted in the reaction chambers. At this time, it was confirmed that only one bead was placed in each reaction chamber with a probability of approximately 100%.

TABLE 2
1 × C buffer (pH = 7.5)

| | | |
|---|---|---|
| Tricine | 50.0 | mM |
| EDTA | 1.0 | mM |
| MgAc | 10.0 | mM |
| DTT | 1.0 | mM |
| PPase | 0.06 | U/mL |
| Tween | 0.1 | % |

Subsequently, a superfluous 1×C buffer other than that in the reaction chambers is removed as much as possible, and then, 5 μL of a reagent for gel containing enzymes described in Table 3 and 15 μL of a 6% solution of photoreactive polyvinyl alcohol (Chemical Formula 1) (BIOSURFINE (registered trademark)-AWP manufactured by Toyo Gosei Co., Ltd.) which is a photoreactive polymer are mixed together and dropped as depicted in FIG. 5B. 502 in FIG. 5B denotes an enzyme-contained photoreactive polymer solution.

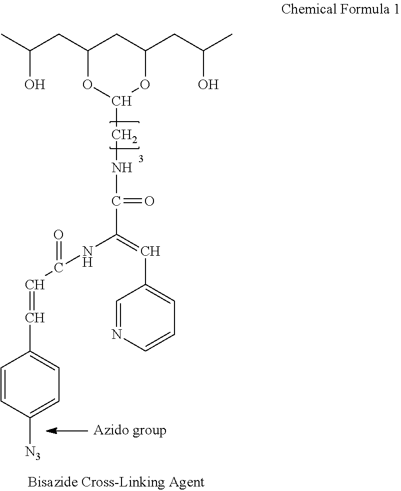

TABLE 3
Reagent for gelling (pH = 7.5)

| | | |
|---|---|---|
| Tricine | 50.0 | mM |
| EDTA | 1.0 | mM |
| MgAc | 10.0 | mM |
| DTT | 1.0 | mM |
| ATPS | 20.0 | U/mL |
| Luciferase | 2000 | GLU/mL |
| Klenow(Exo-) | 0.2 | U/uL |
| SSB | 10.0 | ng/uL |
| Apyrase | 0.1 | mM |
| PPase | 0.06 | mM |

Furthermore, in order to remove a photoreactive polymer other than that inside the reaction chambers, the plate 101 was subjected to spin coating by a spin coater at 500 rpm for 5 seconds and at approximately 5000 rpm for 30 seconds. As a result, as depicted in FIG. 5C, the photoreactive polymer solution on a flat portion other than the reaction chambers 102 can be scattered to the outside of the plate. In this manner, the photoreactive polymer containing enzymes can be charged into only the required portion (inside the reaction chambers) before the curing by the irradiation of ultraviolet light.

Then, drying is performed for 2 minutes, and irradiation of ultraviolet light at 36 W is performed for 1 to 2 minutes, thereby gelling and solidifying the exposed photoreactive polymer at the openings of the reaction chambers. By this means, a form in which the gel 106 containing enzymes is charged into the reaction chambers 102 can be achieved. Note that the 1×C buffer 501 of Table 2 may be immediately dropped to suppress deactivation of the enzyme due to excessive drying.

Next, the top plate 105 is mounted to form the flow cell, and the 1×C buffer in the flow path 109 is displaced with a gel incubation reagent described in Table 4 to perform the incubation for 30 minutes. This was performed in order to introduce polymerase deactivated due to irradiation of ultraviolet light into the DNAs on the beads and to degrade ATP and PPi mixed into the gel. The flow cell after incubation ends is mounted at a predetermined position of the chemiluminescence detecting apparatus depicted in FIG. 3 to perform the pyrosequence.

TABLE 4
Gel incubation reagent (pH = 7.5)

| | | |
|---|---|---|
| Tricine | 50.0 | mM |
| EDTA | 1.0 | mM |
| MgAc | 10.0 | mM |
| DTT | 1.0 | mM |
| Klenow(exo-) | 0.2 | U/uL |
| SSB | 10.0 | ng/uL |
| Apyrase | 0.3 | U/uL |
| PPase | 0.06 | mM |

Here, the photoreactive polyvinyl alcohol (Chemical Formula 1) (BIOSURFINE (registered trademark)-AWP manufactured by Toyo Gosei Co., Ltd.) is used as the photoreactive polymer. Alternatively, another polymer may be used. For example, a photoreactive PEG (Chemical Formula 2) described in Patent Document 6 may be used.

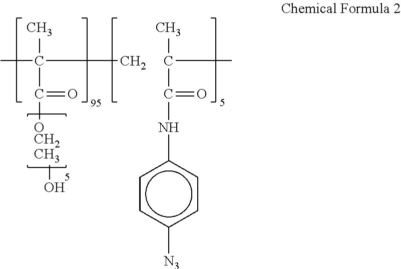

These two photoreactive polymers each contain an azido group (N₃), and a highly-reactive nitrene is produced by the irradiation of ultraviolet light of about 300 nm. This nitrene is reacted with C—H bonding or alkene as a target by the CH insertion or cycloaddition and makes a reaction of polymers to produce a gel, and furthermore, it makes a reaction of the polymers and the enzymes and the polymers and the resin-made plate (the inner walls of the reaction chambers), thereby suppressing drainage of the enzymes and drainage of the entire gel.

However, even these photoreactive polymers cannot be sufficiently gelled without a drying process for about 10 to 30 minutes. In this structure, by using the fact that the enzymes trapped in the reaction chambers are not drained if the photoreactive gel is charged into the concave portions formed in the flow cell and the photoreactive polymer near the surface is sufficiently gelled in the reaction chambers, the drying time is set to be extremely short, that is, 30 seconds to 2 minutes. By this means, deactivation of enzymes such as luciferase due to drying is minimized. Actually, in the case of the drying for 10 minutes, a decrease in activity of luciferase by one to two orders of magnitude was observed.

Polyvinyl pyrrolidone (PVP) (Chemical Formula 3) as another photoreactive polymer and bisazide cross-linking agent as a cross-linking agent can be used, and a photopolymerized polyacrylamido gel can be used.

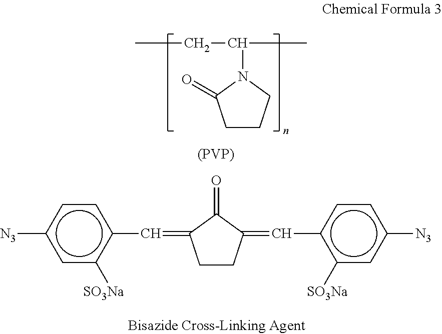

Note that, as described above, when a photoreactive polymer is irradiated with ultraviolet light, the polymers are bonded together to be gelled due to a cross-linking reaction of the azido group. Also at the same time, enzymes such as luciferase and PPDK are reacted with the azido group to be bonded to the polymer molecules. At this time, a part of the enzyme molecules are not boded to the polymer. However, by determining a ratio of the azido group and reaction conditions so that the pore size of the gel is approximately equal to or smaller than the enzyme molecule, the enzymes are prevented from leaking to the outside. On the other hand, for causing a DNA extension reaction, dNTP is supplied near the bead surface by the dNTP diffusion. At this time, the molecular size of dNTP is sufficiently smaller than the size of the enzyme molecule, and therefore is sufficiently smaller than the pore size of the gel. Generally, it is known that a diffusion speed of a molecule smaller than the pore size in a gel is almost unchanged from that in a solution. Actually, in the present embodiment, even if dNTP is supplied through the gel film from outside of the microchamber in the same manner as the conventional art, the DNA extension reaction is completed within several seconds or 10 and a few seconds, it teaches that dNTP is diffused in the gel at a sufficient diffusion speed.

(5) Shape of Minute Reaction Chamber

The shape of the minute reaction chamber 201 is preferably, for example, a columnar shape. By way of examples, a plate fabricated by using a silicon wafer by means of wet etching using masks, a plate fabricated by blaster processing with particles by using a glass such as a slide glass and a plate manufactured by injection molding with a metal mold by using polycarbonate, polypropylene, polyethylene or the like are available. However, these are not meant to be restrictive regarding the material of a minute reactive layer and its fabrication method. In the present embodiment, 110000 reaction chambers are formed on a plate made of polyolefin at intervals of 39 μm by the injection molding.

(6) Example of Luminescence Image

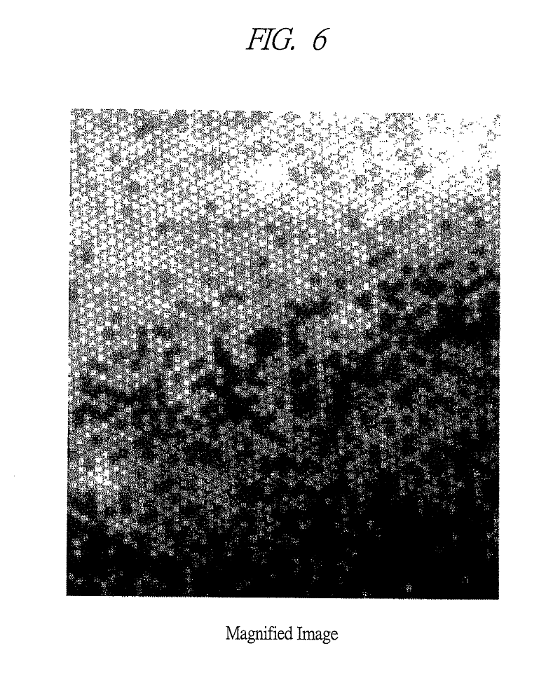

FIG. 6 depicts a luminescence image associated with DNA extension (2-base extension) obtained by the chemiluminescence detecting apparatus described above. One luminescence spot corresponds to one bead, and it can be understood that luminescence can be measured from each bead with sufficient contrast.

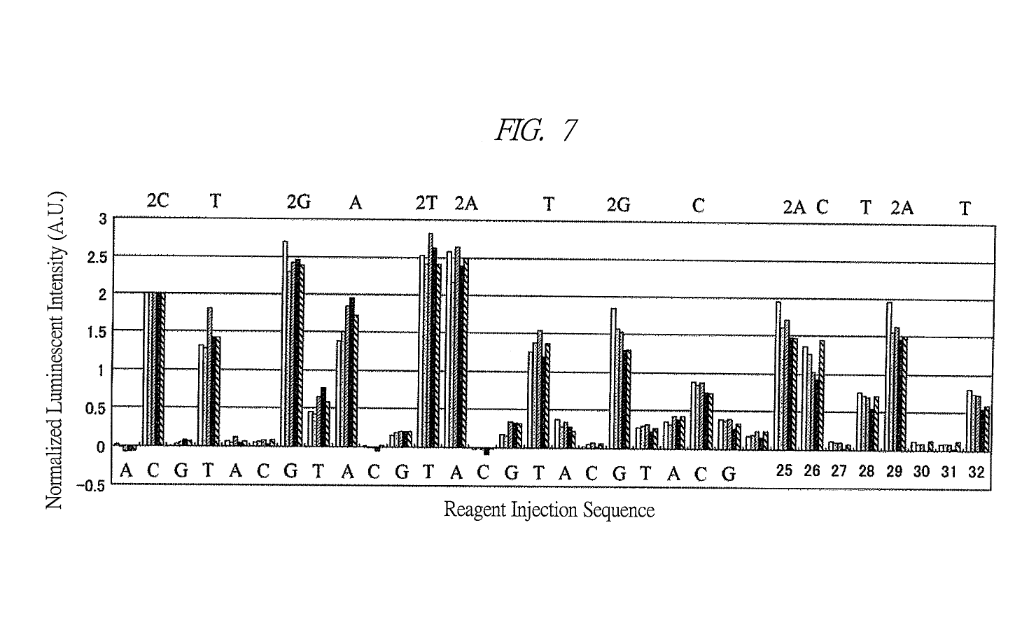

FIG. 7 depicts an example of actual pyrosequencing. In FIG. 7, the horizontal axis represents the type of base caused to flow through the flow cell, and it shows that the reagents are sequentially introduced in the order of A, C, G and T. The vertical axis of FIG. 7 represents luminescent intensity normalized with an initial luminescent intensity. The DNA sequence for use in sequencing is CCTGGATTAATGGCAACTAAT (refer to a sequence listing). Note that the sequence is shown outside the graph.

As can bee seen from FIG. 7, it is confirmed that, when the base matching the sequence is introduced into the flow cell, luminescence occurs with an intensity proportion to the successive base, and also confirmed that pyrosequencing of DNAs immobilized on the beads is possible.

FIG. 8 is a drawing depicting a comparison with a conventional method regarding luminescent intensity. A bar on the left side of FIG. 8 shows the result of measurement of luminescent intensity associated with 1-base extension in the case where a maximum amount of luciferase and ATP sulfrylase is immobilized on microbeads described in Non-Patent Document 2. A bar at the middle shows the result of similar measurement of luminescent intensity in the case where luciferase and ATP sulfrylase are immobilized according to the method of the present invention. With the improvement of an enzyme immobilizing method, a thirty-fold or more improvement in luminescent intensity is confirmed as is understood from the drawing. Furthermore, as shown in a bar on the right side of FIG. 8, it can be understood that a further doubled improvement in sensitivity can be achieved by using PPDK in place of ATP sulfrylase. Note that the same zirconia beads are used for any cases as DNA-immobilized beads, and DNAs of 10⁷ molecules are measured in the immobilized state on the beads.

Note that the beads can also be made small by using the method disclosed herein, details thereof are shown below.

As DNA-immobilized beads, magnetic beads of 4.5 μm are used. The number of DNA molecules is 5×10⁵ per bead. On the flow cell, 1048576 reaction chambers each having a diameter of 6.5 μm are arranged at intervals of 13 μm and a one-to-one correspondence with 1024×1024 pixels of an electron-multiplying-type CCD device is achieved. As a photoreactive polymer, 10 μL of a 6% solution of photoreactive polyvinyl alcohol (Chemical Formula 1) (BIOSURFINE (registered trademark)-AWP manufactured by Toyo Gosei Co., Ltd.) and 10 μL of a reagent for gelling containing enzymes described in Table 3 are mixed together and applied by using a spin coater. Conditions for applying the photoreactive polymer having the enzymes immobilized thereon are 500 rpm for 5 seconds and about 2500 rpm for 30 seconds. By using this, a flow cell is fabricated in the same manner as described above and is mounted on a chemiluminescence detecting apparatus to perform the measurement. Reagent conditions at this time are also the same as those described above. As a result, compared with the case of using the 22 μm beads, the chemiluminescent intensity is decreased to about one fortieth, but the DNA sequencing can be similarly performed.

### Second Embodiment

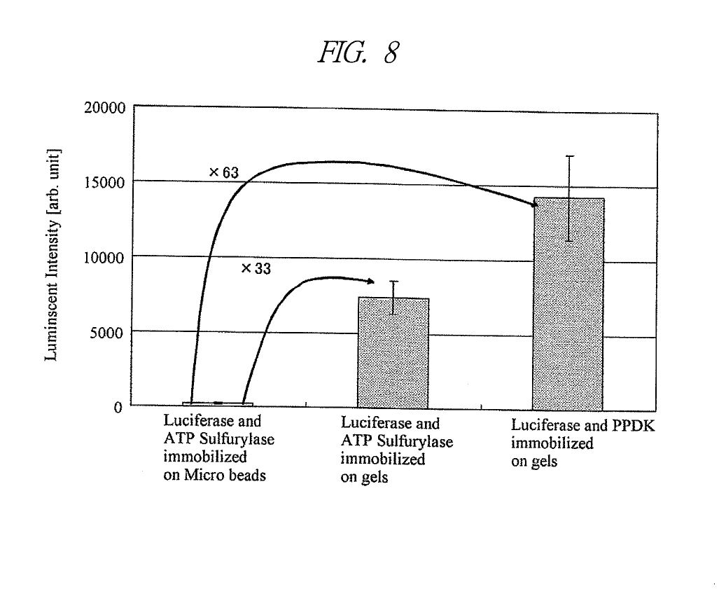

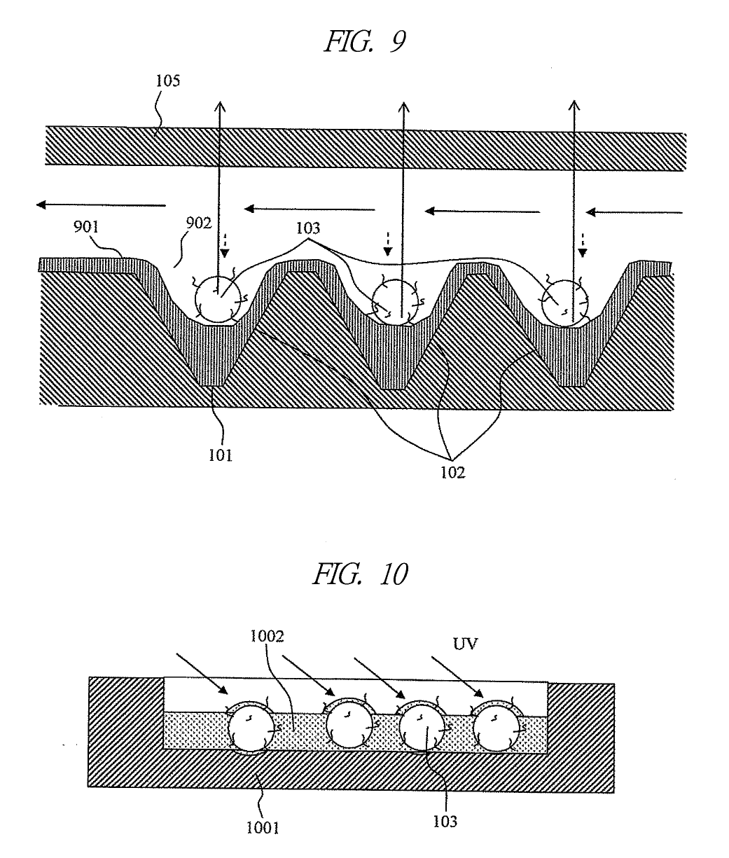

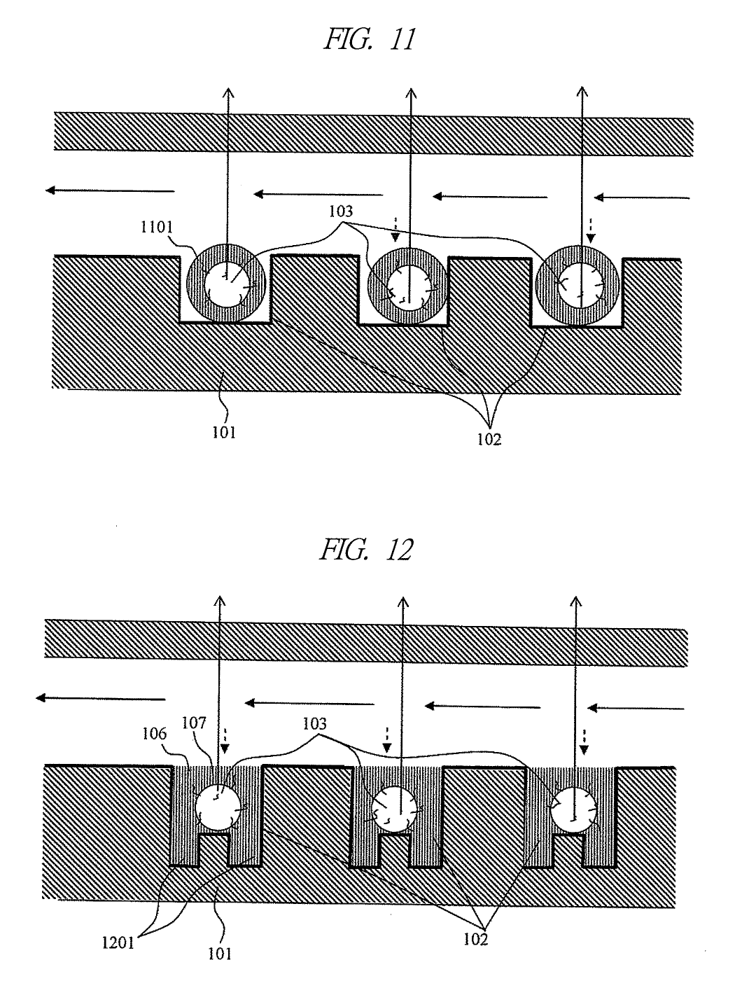

The present embodiment describes an example in which a gel is charged into the reaction chambers and DNA-immobilized beads are then inserted. FIG. 9 depicts a sectional view of the flow cell.

As depicted in FIG. 9, reaction chambers formed by injection molding of polyolefin are two-dimensionally arranged. Here, the reaction chambers 102 have a circular horizontal sectional shape, and have a shape of a frustum whose diameter of entrance is larger, that is, 45 μm and diameter of bottom is smaller, that is, 5 μm. Although the diameter of the bottom is set at 5 μm considering a problem of processing accuracy here, the diameter may be 0. In this case, the reaction chamber has not a frustum shape but a cone shape. The depth of the reaction chamber is 60 μm.

In this state, 15 μL of a 6% solution of photoreactive polyvinyl alcohol (Chemical Formula 1) (BIOSURFINE (registered trademark)-AWP manufactured by Toyo Gosei Co., Ltd.) and 5 μL of a reagent for gelling containing the enzymes described in Table 3 are mixed together and subjected to spin coating at 500 rpm for 5 seconds and at 5000 rpm for 30 seconds.

As a result, a photoreactive polymer solution can be applied in a shape as depicted in FIG. 9. Then, the solution is dried for 2 minutes and irradiated with ultraviolet light of 36 W for 1 to 2 minutes. By this means, a gel layer 901 containing the enzymes is formed. In this state, many plates on which the enzyme-immobilized gel layer 901 is formed can be manufactured. By forming a concave portion 902 of this plate having a depth approximately equal to the diameter of the DNA-immobilized bead (1 to 1.7 times larger than the bead diameter), one DNA-immobilized bead can be charged into each reaction chamber 102.

By forming a flow cell using the plate where this gel layer is formed, pyrosequence can be performed with an apparatus similar to that of the first embodiment. Also in the present embodiment, another substance as shown in (Chemical Formula 2) or (Chemical Formula 3) may be used as a photoreactive gel.

Since a contact area between the beads 103 inserted in the reaction chambers 102 and the gel according to the present embodiment is smaller compared with the case of the first embodiment described above, reactive efficiency is inferior. However, since a flow cell can be provided to users in a state where a gel is charged therein, the trouble of the users in preparing an experiment can be reduced and the user usability can be improved.

### Third Embodiment

The present embodiment describes an example in which DNA-immobilized beads are formed in a shape of being wrapped with a gel and these beads are charged into reaction chambers, thereby performing the chemiluminescence measurement.

A resin-made shallow container 1001 having a depth of about 1 mm is filled with a solution having 100 μL of a 6% solution of photoreactive polyvinyl alcohol (Chemical Formula 1) (BIOSURFINE (registered trademark)-AWP manufactured by Toyo Gosei Co., Ltd.) and 100 μL of a reagent for gelling containing the enzymes described in Table 3 above mixed together. Here, DNA-immobilized zirconia-made beads 102 are injected, and as depicted in FIG. 10, ultraviolet light (36 W) is applied diagonally as indicated by arrows for 1 minute while vibrations are applied at 500 rpm so that the zirconia beads are partly exposed from the solution, thereby forming a gel film having a thickness of about 5 μm only on the surfaces of the DNA-immobilized beads.

Then, beads 1101 having their surfaces coated with the gel containing enzymes are inserted in a plate having two-dimensionally arranged reaction chambers with polyolefin. FIG. 11 depicts an inserted state. Here, reaction chambers having a depth approximately equal to the diameter of the beads coated with the gel are formed.

By using these beads and plate, pyrosequence can be performed with an apparatus similar to that of the first embodiment. Note that, also in the present embodiment, other substances such as those represented in Chemical Formula 2 and Chemical Formula 3 may be used as the photoreactive gel.

### Fourth Embodiment

The present embodiment describes an example of a chemiluminescence detecting device or apparatus in which a further improvement in sensitivity is achieved by using reaction chambers whose area capable of charging a gel containing enzymes is increased.

A protrusion having a diameter of about 5 μm and a height of about 10 μm is provided at a bottom portion of a resin-made reaction chamber. The diameter of the reaction chamber is, for example, 30 μm, and the depth thereof at the deepest portion is set at 40 μm and the depth at the shallowest portion near the center is set at 30 μm. By forming reaction chambers each having the shape as described above, only one DNA-immobilized bead having a diameter of 22 μm can be charged even when the depth at the deepest portion is made deeper.

In this manner, by forming areas such as areas 1201 where a liquid photoreactive polymer can be charged but a spherical bead cannot be charged, the volume of the gel chargeable to one reaction chamber can be increased. More specifically, enzyme activity required for chemiluminescence per reaction chamber can be improved. Thus, it is possible to prevent pyrophosphoric acid (PPi) from being discharged outside the reaction chambers or it is possible to reduce the amount of pyrophosphoric acid to be discharged (loss of pyrophosphoric acid can be reduced).

1. A DNA detecting apparatus which detects chemiluminescence from a plurality of reaction chambers for DNA detection or DNA analysis, the apparatus comprising:
a flow cell having a plate on a surface of which a plurality of reaction chambers are one-dimensionally or two-dimensionally arranged; and
light detecting means having a plurality of pixels and detecting chemiluminescence,
wherein one or plural beads having up to one type of DNA immobilized thereto and a gel containing at least an enzyme required for chemiluminescence detection are charged into each of the reaction chambers.

2. The DNA detecting apparatus according to claim 1,
wherein the gel charged into the reaction chamber is gelled and solidified by light irradiation at an optical opening of the reaction chamber.

3. The DNA detecting apparatus according to claim 1,
wherein the enzyme contained in the gel includes luciferase.

4. The DNA detecting apparatus according to claim 3,
wherein the gel charged into the reaction chamber contains an azido group in a polymer gelled by light irradiation.

5. The DNA detecting apparatus according to claim 1,
wherein each of the reaction chambers has a convex structure therein, only one DNA-immobilized bead can be charged into the reaction chamber, and an area where the gel can be charged is present inside the reaction chamber.

6. A DNA detecting device used for detecting chemiluminescence from a plurality of reaction chambers, the device comprising:
a flow cell having a plate on a surface of which a plurality of reaction chambers are one-dimensionally or two-dimensionally arranged,
wherein a gel containing an enzyme required for the detection of chemiluminescence is charged in the reaction chambers, and
in a state where the gel is charged, a space (concave portion) for charging a DNA-immobilized bead remains inside the reaction chamber at a portion closer to an opening of the reaction chamber than to a portion where the gel is charged.

7. The DNA detecting device according to claim 6,
wherein the space has a depth approximately equivalent to a diameter of the bead.

8. The DNA detecting device according to claim 6,
wherein the gel charged into the reaction chamber is gelled and solidified by light irradiation at the opening of the reaction chamber.

9. The DNA detecting device according to claim 6,
wherein the enzyme contained in the gel includes luciferase.

10. The DNA detecting device according to claim 8,
wherein the gel charged into the reaction chamber contains an azido group in a polymer gelled by light irradiation.

11. The DNA detecting device according to claim 6,
wherein the reaction chamber formed on the plate has a tapered shape whose opening is wider than a bottom portion.

12. The DNA detecting device according to claim 6,
wherein each of the reaction chambers has a convex structure therein, only one DNA-immobilized bead can be charged into the reaction chamber, and an area where the gel can be charged is formed inside the reaction chamber.

13. A DNA detecting method which detects chemiluminescence from a plurality of reaction chambers for DNA detection or DNA analysis, the method comprising the steps of:
charging DNA-immobilized beads into a plurality of reaction chambers which are one-dimensionally or two-dimensionally arranged on a plate;
charging a photoreactive polymer containing an enzyme required for chemiluminescence detection into the reaction chambers;
gelling the photoreactive polymer by light irradiation; and
introducing a reagent relating to a luminescence reaction onto the reaction chambers to cause chemiluminescence and detecting this chemiluminescence.

14. The DNA detecting method according to claim 13, further comprising the step of:
removing the photoreactive polymer remaining in a portion other than the reaction cambers on the plate,
wherein, after the superfluous photoreactive polymer is removed, the step of gelling the photoreactive polymer by the light irradiation is performed.

15. The DNA detecting method according to claim 14,
wherein the step of charging the photoreactive polymer into the reaction chambers and the step of gelling the photoreactive polymer by light irradiation are performed a plurality of times.

16. A DNA detecting method which detects chemiluminescence from a plurality of reaction chambers for DNA detection or DNA analysis, the method comprising the steps of:
charging a photoreactive polymer containing an enzyme into a plurality of reaction chambers which are one-dimensionally or two-dimensionally arranged on a plate;
gelling the photoreactive polymer by light irradiation;
in a state where the gel is charged, charging DNA-immobilized beads into a space (concave portion) inside the reaction chamber at a portion closer to an opening of the reaction chamber than to a portion where the gel is charged; and
introducing a reagent on the reaction chambers to cause chemiluminescence and detecting this chemiluminescence.

17. The DNA detecting method according to claim 16,
wherein the step of charging the photoreactive polymer into the reaction chambers and the step of gelling the photoreactive polymer by light irradiation are performed a plurality of times.


SEQUENCE LISTING (2 sequences)

SEQ ID NO: 1 - DNA, 21 bp, Artificial Sequence
  Description of Artificial Sequence Synthetic oligonucleotide
cctggattaatggcaactaat

SEQ ID NO: 2 - DNA, 23 bp, Artificial Sequence
  Description of Artificial Sequence Synthetic oligonucleotide
acgtacgtacgtacgtacgtacg